(19) United States
(12) Patent Application Publication
(10) Pub. No.: US 2011/0067706 A1
(43) Pub. Date: Mar. 24, 2011

(54) Male sheath ejaculation cap

(76) Inventor: Minh Cong Dang, Anaheim, CA (US)

(21) Appl. No.: 12/586,200

(22) Filed: Sep. 18, 2009

(51) Int. Cl. A61F 6/04

(52) U.S. Cl. 128/844

(57) ABSTRACT

The Ejaculation Cap is a containment chamber and receptacle for semen from the penis resulting from sexual activity. The device is tubular with a closed end and an open end with the body of the device to covering the length of the penis. The closed end can be flat, circular, or gusseted. The material used can be of some soft, flushable, disposable or lubricated tissue or washable fabric. The construction can be seamed or seamless and made to fit different sized penises. The purpose of the device is to enhance cleanliness, but construction and/or materials can include features that also enhance pleasure.

### CROSS-REFERENCE TO RELATED APPLICATIONS

(Not Applicable)

### STATEMENT REGARDING FEDERALLY SPONSORED RESEARCH AND DEVELOPMENT

(Not Applicable)

### NAMES OF PARTIES TO A JOINT RESEARCH AGREEMENT

(Not Applicable)

### STATEMENT REGARDING REFERENCE TO SEQUENCE LISTINGS, A TABLE OR A COMPUTER PROGRAM LISTING COMPACT DISC APPENDIX

(Not Applicable)

### BACKGROUND OF THE INVENTION

1. Field of Invention

This invention relates to the field of male penis coverings. More specifically, the invention relates to a sheath that covers the male penis during the ejaculation of semen. Said sheath may or may not be constructed to enhance sexual pleasure.

2. Related Art

Many types of male undergarments that offer protection for the penis exist in prior art. U.S. Pat. No. 2,888,014 describes a suspensory with a pouch that supports the penis and scrotum. U.S. Pat. No. 1,686,943 depicts an athletic supporter that protects the penis and scrotum while providing a means for recording a striking blow to this area of the body. U.S. Pat. No. 850,298 delineates a suspensory that provides protective pouches for both the penis and the scrotum. U.S. Pat. No. 5,237,706 describes a male garment with a scrotal pouch that repositions both the penis and the scrotum to a more prominent anterior position. U.S. Pat. No. 796,278 depicts a supporter for the scrotum to be used during the treatment of a number of male diseases. U.S. Pat. No. 3,550,590 describes a pouch-like surgical bandage to protect the penis and/or scrotum after injury or surgery. U.S. Pat. No. 5,735,837 depicts a urine-absorbent bag for use as a urine collection device to provide a bather to wetness. The problems associated with these related designs are that they are designed for use as either a bandage, support, or underwear.

U.S. Pat. No. 4,981,147 details a disposable, unitary coital protective garment, comprising a groin-covering panel and an integrally formed penile sheath for the purpose of protecting both partners from sexually transmitted diseases. U.S. Pat. No. 4,601,716 depicts a sanitary sheath for males who tend to dribble urine. The problems associated with these related designs are that they are designed for use as either a contraceptive or a sanitary pad for use as a remedy for incontinence. None of these purposes are conducive to promoting sexual pleasure and all of them have a specific purpose other than merely containing ejaculate for the purpose of providing an inexpensive, clean, stimulating vehicle to contain semen associated with masturbation.

Also, the overall design for use, disposability and manufacturing costs and design separate the prior art from this invention. Construction of prior and related art does not provide specifically for the stimulation of the penis associated with sexual activity.

### BRIEF SUMMARY OF THE INVENTION

The object of the invention is to provide a hygienic, disposable, discreet, natural, easy to use, cost effective, and pleasurable sexual experience for the user by providing a covering of the penis during sexual excitement for the purpose of fully containing ejaculate. The design of this invention provides a one-piece garment with clean, simple lines to accommodate the natural changes in size and shape of the male penis during sexual activity. The device promotes hygiene by causing a barrier between the penis's orifice and exterior surroundings.

The material from which the ejaculation cap is made can vary greatly, depending upon desired pleasure sensation and disposability. The device is simple and can be constructed from a variety of materials ranging in cost and textures, including, but not limited to, soft cloths, paper toweling, wax coated paper or septic safe materials similar to toilet tissues. The purpose of the device is to eliminate the messiness and odors normally related to ejaculation.

### BRIEF DESCRIPTION OF THE SEVERAL VIEWS OF THE DRAWINGS

The drawings depict a specific use for the ejaculation cap as a penis covering for the purpose of preventing the messiness associated with ejaculation. The purpose may also be to enhance pleasure depending upon material(s) used in construction and/or lubricant(s) added to the material(s).

The drawings depict design(s) that can be straight, rounded, or gusseted at the closed end, and include a depiction of how the ejaculation cap is used.

FIG. 1 shows the circular open end, that is the same for all three closed end designs. It also shows the flat closed end design.

FIG. 2 shows the rounded closed end design.

FIG. 3 depicts the method of use regardless of closed end design.

FIG. 4 depicts a frontal view of the use and also the gusseted closed end design.

FIG. 5 shows the view of the rounded closed end design option.

### DETAILED DESCRIPTION OF THE INVENTION

The purpose of the invention is to provide a receptacle to contain ejaculate following masturbation or other sexual activity. The invention is intended to be discarded after a single use, but may be cleaned for re-use depending upon design sophistication and materials used. Lubricant(s) and/or medications may be applied to the interior surface.

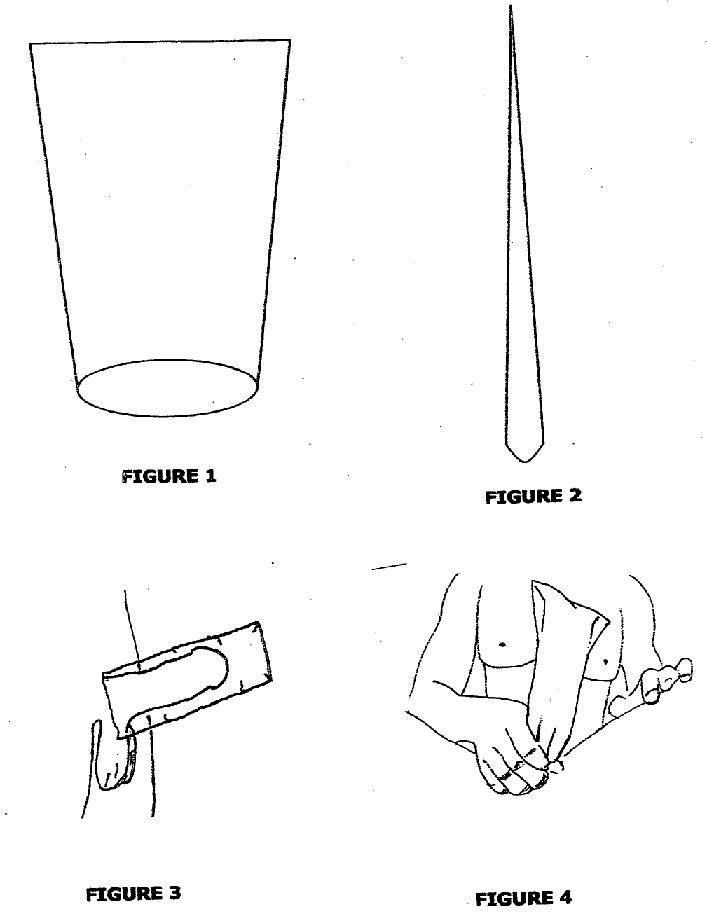

The means for construction may be simple or complex. Simple construction may be by taking a rectangular material and folding it crosswise and closed on both lengths so one end is closed, on the fold, and the opposite end left open as shown in FIG. 1.

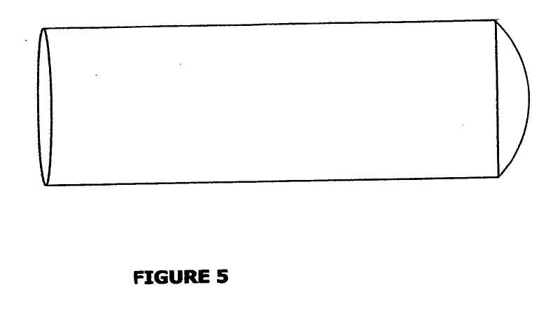

More complex construction includes creating a rounded closed end, as shown in FIGS. 2 and 5 or a gusseted pocket at the closed end, as shown in FIG. 4.

Materials can be any common soft tissue, toweling, or a material specially designed for this use.

To use, insert the male penis through the open end, covering it during sexual stimulation activity as shown in FIGS. 3 and 4. Upon ejaculation, the semen will be captured by the sheath, which can either be discarded, flushed in the toilet, or cleaned for re-use.

The most economical mode is to produce the invention from disposable materials. The best mode is to construct each sheath from material(s) designed to enhance sensation. Either mode can be constructed with any design.

What is claimed is:

1) A penis sheath and receptacle to receive ejaculate, comprising:
a. an open end portion through which the male penis is inserted,
b. a middle portion surrounding and enclosing the penis; and
c. a sealed/closed end portion to contain the ejaculate.

2) Said sheath can vary in construction to be:
a. lubricated,
b. flat, rounded, or gusseted at the closed end,
c. in different sizes to accommodate penis length and circumference, and/or
d. medicated.

3) A penis sheath that can be modified to enhance sexual pleasure, comprising:
a. lubrication added to the material;
b. construction features that increase stimulation;
